(19) United States
(12) Patent Application Publication
(10) Pub. No.: US 2003/0118536 A1
(43) Pub. Date: Jun. 26, 2003

(54) Topical compositions and methods for treatment of adverse effects of ionizing radiation

(76) Inventor: Richard A. Rosenbloom, Elkins Park, PA (US)

Correspondence Address: KNOBLE & YOSHIDA, EIGHT PENN CENTER, SUITE 1350, 1628 JOHN F KENNEDY BLVD, PHILADELPHIA, PA, 19103, US

(21) Appl. No.: 10/288,761

(22) Filed: Nov. 6, 2002

Related U.S. Application Data
(63) Continuation-in-part of application No. 10/132,642, filed on Apr. 25, 2002.
Application No. 10/132,642 is a continuation-in-part of application No. 10/045,790, filed on Jan. 14, 2002.
Application No. 10/045,790 is a continuation-in-part of application No. 09/993,003, filed on Nov. 6, 2001.

(51) Int. Cl. A61K 7/06

(52) U.S. Cl. 424/70.1

(57) ABSTRACT

Compositions and methods for the prevention, reduction or treatment of adverse effects due to exposure to ionizing radiation, including at least one flavonoid and at least one non-flavonoid antioxidant, optionally formulated in a acceptable carrier for a topical composition. The composition of the present invention may further include optional ingredients such as selenium, selenium compounds, anti-inflammatories, organic germanium compounds, compounds that regulate cell differentiation, Korean ginseng, American ginseng, Siberian ginseng and B-complex vitamins. A method for the topical administration of the composition in accordance with the present invention for the purpose of reducing, treating or preventing adverse effects caused by ionizing radiation involves topically administering a safe and effective amount of the composition of the invention an area of skin, which has been, is being or will be exposed to ionizing radiation. The compositions and methods can be employed to reduce, treat or prevent radiation injury caused by a wide variety of types of exposure to ionizing radiation.

### CROSS-REFERENCE TO RELATED APPLICATIONS

The present application is a continuation-in-part of U.S. patent application Ser. No. 10/132,642, filed on Apr. 25, 2002, which is a continuation-in-part of U.S. patent application Ser. No. 10/045,790, filed on Jan. 14, 2002, which is a continuation-in-part of U.S. patent application Ser. No. 09/993,003, filed on Nov. 6, 2001, each of which is incorporated herein by reference in its entirety.

### BACKGROUND OF THE INVENTION

1. Field of the Invention

The present invention relates to topical compositions and methods for treatment of adverse effects of ionizing radiation. More specifically, the present invention relates to topical compositions and methods for treatment of adverse effects of ionizing radiation employing a safe and effective amount of at least one flavonoid and at least one non-flavonoid antioxidant.

2. Description of the Prior Art

It is generally known that extensive radiation exposure or exposure to ionizing radiation may cause radiation injury. Radiation injury may range from less serious injuries such as radiation dermatitis to more serious injuries such as those causing vomiting, bone marrow failure, intestinal death and/or instant incineration. Ionizing radiation can adversely affect the appearance of the skin and/or hair. Adverse effects on the appearance of the skin and/or hair caused by ionizing radiation can include deterioration of the appearance of skin such as, for example, redness, discoloration, dryness of the skin. Such injuries or damage may be caused by radiation emitted from x-rays such as those used in diagnostic equipment, γ-rays such as those emitted from radioactive materials or from numerous other sources.

Many attempts have been made to reduce, control or cure radiation injury. U.S. Pat. No. 5,543,140 to Nakai et al discloses a method of preventing or inhibiting radiation injury by administering interleukin-1-α derivatives. In particular, Nakai et al uses an interleukin-1-α modified by replacing the Asn at the 36 position with Asp, and replacing the Cys at the 141 position with Ser. The modified interleukin-1-α derivative is preferably produced using recombinant DNA techniques, which are complicated and burdensome. In addition, the potential adverse side effects of the modified Interleukin-1-α derivatives are not well known.

U.S. Pat. No. 5,767,092 to Koezuka et al. discloses a composition, which may be therapeutically or prophylactically useful in promoting bone marrow cell proliferation and protecting human bone marrow cells against radiation damage. The composition disclosed in Koezuka et al. contains α-galactosylceramide. However, radiation may cause other injuries in addition to damage to bone marrow cells and thus this composition has limited applicability.

There still remains a need in the art for effective compositions and methods to prevent, reduce and treat adverse effects of ionizing radiation.

Accordingly, it is an objective of certain embodiments of the present invention to provide a topical composition that, when applied, will reduce, treat or prevent adverse effects of ionizing radiation.

It is further objective of certain embodiments of the present invention to provide methods to effectively reduce, treat or prevent adverse effects of ionizing radiation by topical application of a composition according to the invention.

These and other objects of the present invention will be apparent from the summary and detailed descriptions of the invention, which follow.

### SUMMARY OF THE INVENTION

In a first aspect, the present invention relates to topical composition for reducing, treating or preventing at least one adverse effect of ionizing radiation, such as radiation injury. The topical composition includes a mixture of at least two ingredients, a non-flavonoid antioxidant and at least one flavonoid, formulated in an acceptable carrier for a topical composition.

In a second aspect, the present invention relates to a method of administering a topical composition for the reduction, treatment or prevention of at least one adverse effect of ionizing radiation, such as radiation injury. In the method, an effective amount of a mixture of at least two ingredients, a non-flavonoid antioxidant and at least one flavonoid, formulated in an acceptable carrier for a topical composition, is applied topically to a mammal at risk for radiation exposure, to a mammal being exposed to radiation or to a mammal that has already been exposed to ionizing radiation to reduce, treat or prevent at least one adverse effect of ionizing radiation, such as radiation injury.

### DETAILED DESCRIPTION OF THE PREFERRED EMBODIMENTS

As used herein, an “acceptable” component is one that is suitable for use with humans and/or animals without undue adverse side effects (such as toxicity, irritation, and allergic response) commensurate with a reasonable benefit/risk ratio. Further, as used herein, the term “safe and effective amount” refers to the quantity of a component, which is sufficient to yield a desired therapeutic response without undue adverse side effects (such as toxicity, irritation, or allergic response) commensurate with a reasonable benefit/risk ratio when used in the manner of this invention. The specific “safe and effective amount” will, obviously, vary with such factors as the particular condition being treated, the physical condition of the patient, the duration of the treatment, the nature of concurrent therapy (if any), and the specific formulations employed.

Adverse effects of ionizing radiation may include radiation injury or damage to any part of the body of a mammal including humans caused by exposure to radiation. Such injury or damage may include local effects, for example, radiation dermatitis, and systemic effects, for example, bone marrow cell damage, intestinal damage, and symptoms or conditions such as cancer, and DNA mutation that may be caused either directly or indirectly, by exposure to one or more ionizing radiations such as fluoroscopic radiation, ultraviolet radiation, proton radiation, alpha radiation, beta radiation, x-ray radiation and gamma radiation. Exposure to ionizing radiation may occur from therapeutic or medical uses of radiation or from accidental or malicious exposure to radiation. Ionizing radiation can disrupt DNA molecules in living cells and cause mutation, damage, and/or death of the living cells, which in turn may result in cancer and genetic mutation. In addition, ionizing radiation can also cause changes in the chemical balance of cells, which may further cause cancer.

In one embodiment, the compositions and methods of the present invention may be employed to treat radiation injury resulting from exposure to one or more ionizing radiations. Ionizing radiation is any form of radiation that has enough energy to knock electrons out of atoms or molecules, thereby creating ions. Commonly, ionizing radiation includes proton radiation, alpha radiation, beta radiation, x-ray radiation, gamma radiation and neutron radiation. Ionizing radiation may further include cosmic radiation that penetrates the Earth's atmosphere from space and which consists mainly of protons, alpha particles, and heavier atomic nuclei. Positrons, mesons, pions, and other exotic particles can also be found in ionizing radiation. In another embodiment, the one or more adverse effects of radiation being reduced, treated or prevented using a method and/or a composition of the present invention is caused by one or more of alpha and beta particle radiation, gamma ray radiation and x-ray radiation.

Alpha and beta particles and gamma rays can come from natural sources or can be technologically produced. Natural radiation comes from cosmic rays, naturally occurring radioactive elements found in the earth's crust (uranium, thorium, etc.), and radioactive decay products such as radon and its subsequent decay products. In addition to these natural sources, radiation can come from such wide-ranging sources as hospitals, research institutions, nuclear reactors and their support facilities, certain manufacturing processes, and facilities involved in nuclear weapons production. Radiation can further be a result of a nuclear power plant accident, a nuclear attack by a nuclear or “dirty” bomb, and/or an accidental nuclear material leakage.

The invention is particularly useful for mammals that are, or will be, engaging in activities involving a high risk of radiation exposure. Also, the invention can be employed to treat mammals exposed to radiation as a result of a radiation attack, a nuclear accident, radiation from diagnostic instruments and therapeutic radiation used to treat, for example, cancer. The adverse effect of radiation reduced, treated or prevented by the compositions and methods of the present invention may be caused by exposure to non-therapeutic ionizing radiation, such as, for example, accidental radiation exposure, exposure to radioactive materials released by nuclear attack or nuclear accidents, and exposure to diagnostic instruments such as an x-ray machine, a CT-scan, or a synchrotron, all of which employ radiation. Alternatively, the adverse effect of radiation injury prevented, reduced or treated by the compositions and methods of the present invention may be caused by exposure to therapeutic radiation, such as radiation therapy used in cancer treatment.

The Topical Composition

In a first aspect, the present invention relates to a topical composition for reducing, treating or preventing at least one adverse effect of ionizong radiation. In this aspect of the invention, the topical composition includes a mixture of at least two ingredients: a non-flavonoid antioxidant and at least one flavonoid, formulated in an acceptable carrier for a topical composition.

The flavonoids may have radioprotective effects. In addition, flavonoids may have other beneficial effects such as acting as an anti-inflammatory and maintaining the structural integrity of ischemic or hypoxic tissue, which may occur after radiation exposure. Examples of flavonoids include, without limitation, flavonones, flavanols, anthocyanidins, proanthocyanidins, procyanidolic oligomers, biflavans, rutinosides, hydroxyethylrutinosides, and leucoanthocyanins. The choice of specific flavonoids to be included in the composition may be determined by factors such as toxicity, bioavailability, solubility or dispersability, and the like. Preferred flavonoids are derived from natural sources such as plants. Also, flavonoids include compounds derived from known flavonoids, which compounds retain at least some of the desired activity of the flavonoid from which it is derived.

Examples of flavonoids suitable for use in the present invention include, without limitation, 1,2,3,6-tetra-o-gallyol-β-d-glucose; 2′-o-acetylacetoside; 6,3′,4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; catechins; chrysin; chrysin-5,7-dihydroxyflavone; chrysoeriol; chrysosplenol; chrysosplenoside-a; chrysosplenoside-d; cosmosiin; δ-cadinene; curcumin; cyanidin; dihydroquercetin; dimethylmussaenoside; diacerylcirsimaritin; diosmin; diosmetin; dosmetin; ebinin; epicatechin; ethyl brevifolin carboxylate; flavocannibiside; flavosativaside; galangin; genistein; ginkgo flavone glycosides; ginkgo heterosides; gossypetin; gossypetin-8-glucoside; haematoxylin; hesperidine; hispiduloside; hyperin; indole; iridine; isoliquiritigenin; isoliquiritin; isoquercitrin; jionoside; juglanin; kaempferol; kaempferol-3-rhamnoside; kaempferol-3-neohesperidoside; kolaviron; licuraside; linariin; linarin; lonicerin; luteolin; luteolin-7-glucoside; luteolin-7-glucoronide; macrocarpal-a; macrocarpal-b; macrocarpal-d; macrocarpal-g; maniflavone; morin; methyl scutellarein; monoHER, diHER, triHER, tetraHER, myricetin; naringenin; naringin; nelumboside; nepetin; nepetrin; nerolidol; oligomeric proanthocyanidins; oxyayanin-a; pectolinarigenin; pectolinarin; pelargonidin; phloretin; phloridzin; quercetagetin; quercetin; quercimertrin; quercitrin; quercitryl-2″ acetate; reynoutrin; rhamnetin; rhoifolin; rutin; scutellarein; sideritoflavone; silibin; silydianin; silychristine; silymarin; sophoricoside; sorbarin; spiraeoside; taxufolin; trifolin; vitexin; and wogonin, and the pharmaceutically acceptable salts; solvates; and derivatives of these compounds.

Another preferred flavonoid source is green tea extract, and exemplary compounds contained therein such as, for example, (-)-epigallocatechin-3-gallate, (-)-epigallocatechin-3-gallate, (-)-epigallocatechin and/or (-)-epicatechin. Studies (see Elmets, C. A. et al, J. Am. Acad. Dermatol., 44 (3); 425-32, March, 2001) have shown that green tea extract is effective in inhibiting erythema. The at least one component of green tea or green tea extract is used in an amount of about 250 mg-2 gm, more preferably of about 400-1.5 gm, and most preferably of about 500 mg-1 gm, per one pound of the topical base.

The flavonoid used in the composition of the present invention may also include one or more curcuminoids. Examples of curcuminoids include, without limitation, curcumin (diferuloylmethane), desmethoxycurcumin (hydroxycinnamoyl feruloylmethane), and/or bis-desmethoxycurcumin (dihydroxydicinnamoyl methane) (see Drug Analysis by Chromatography and Microscopy, p. 169, Ann Arbor Science Inc., 1973). These compounds may be purchased from commercial sources or isolated from turmeric. Methods for isolating curcuminoids from turmeric are known, (see Janaki and Bose, An Improved Method for the Isolation of Curcumin From Turmeric, J. Indian Chem. Soc. 44:985 (1967)). Alternatively, curcuminoids for use in the present invention can be prepared by synthetic methods. Curcuminoids possess antioxidant properties and also have anti-inflammatory, anti-tumor and other valuable properties.

More preferred flavonoids are quercetin, rutin, myricetin, kaempferol, myrecetrin, flavonoid components of green tea, and curcuminoids. Quercetin, rutin and flavonoid components of green tea are the most preferred flavonoids for use in the composition of the present invention. The aforementioned compounds may have some anti-inflammatory activity and/or may help stabilize cell membranes in combination with a relatively low toxicity, both of which activities may be beneficial in the treatment of radiation. Also, pharmaceutically acceptable salts of these flavonoids may be employed. The particular flavonoid or flavonoids included in the composition may be determined by factors such as toxicity, bioavailability, solubility or dispersability, among others.

The particular flavonoids mentioned above are also preferred since some of these compounds may provide additional beneficial effects in the composition of the present invention. For example, quercetin may also have antioxidative and/or anticlastogenic effects. It may prevent the decrease of endogenous ascorbic acid (vitamin C) in bone marrow after gamma-ray irradiation. In addition, some of the flavonoids may act as a radical scavenger to scavenge free radicals such as hydroxyl radicals to enhance their radioprotective effects.

The at least one flavonoid of the present invention is administered in a safe and effective amount. Every pound of a preferred topical composition of the present invention preferably includes about 1 to about 150 grams of one or more flavonoids, about 0.1 to about 50 grams of non-flavonoid antioxidants, and other suitable ingredients such as topical carriers.

Preferably, the flavonoid is used in an amount of about 2 to about 100 grams per pound of the composition. More preferably, the flavonoid is employed in an amount of about to about 10-50 grams per pound of the composition, and, still more preferably, about 15 to about 40 grams per pound of the composition.

In a preferred embodiment, about 10 g/pound of quercetin is used. In another preferred embodiment, about 5 g/pound to about 25 g/pound, more preferably about 5 g/pound, of rutin are added to the composition.

Non-Flavonoid Antioxidants

One ingredient in the topical composition of the present invention is at least one non-flavonoid antioxidant. The non-flavonoid antioxidant may be a single compound or material or a mixture of two or more compounds and/or materials. Compounds and materials which may be used as non-flavonoid antioxidants are those which exhibit antioxidant activity when administered to a patient in a safe and effective amount to provide sufficient antioxidant activity, and which do not react with one or more of the ingredients of the composition resulting in a substantial loss of activity of one or more of the ingredients. Preferred non-flavonoid antioxidants are those that occur naturally in the human body and/or materials obtained from plants or animals, or derivatives thereof.

Preferred non-flavonoid antioxidants are selected from ascorbic acid (vitamin C), its esters, for example, ascorbyl palmitate, and other compounds having vitamin C activity such as those generally called Ester-C (tm) that are disclosed in U.S. Pat. Nos. 4,822,816 and 5,070,085; vitamin A and its esters, for example, vitamin A palmitate; vitamin E and its esters, for example, vitamin E acetate; lipoic acid, preferably α-lipoic acid, and more preferably DL-α-lipoic acid; carotenoids such as β-carotene; chlorophyllin and its salts; coenzyme Q10; glutathione; L-dopa, cysteine, N-acetyl cysteine, cystine, pangamic acid (dimethyl glycine), taurine, tyrosine, carbohydrates such as beta-1,3-glucan, germanium, alpha-hydroxy acids including glycolic acid and lactic acid, phytic acid (inositol hexaphosphate), caffeic acid (3,4-dihydroxy-cinnamic acid), ellagic acid, 3,3′,4-tri-o-methyl ellagic acid, ferulic acid, gallic acid, gamma-oryzanol, resveratrol (trans-3,5,4′-trihydroxystilbene), zingerone (4-(4-hydroxy-3-methoxyphenyl)-2-butanone), nordihydroguaiaretic acid, pyrroloquinoline quinone, allicin, dithiolthiones, glucosilinates, S-allyl-L-cysteine, tocotrienols, carnosol, indole, polyphenols, including those derived from green tea extract, and the pharmaceutically acceptable salts, solvates, and derivatives thereof which exhibit antioxidant activity.

In one embodiment of the invention disclosed herein mixtures of two or more non-flavonoid antioxidants are employed in the composition.

Antioxidant mixtures optionally contain one or more of vitamin A, vitamin E acetate, vitamin D₃ and ascorbyl palmitate (vitamin C). The antioxidants may be used in their pharmaceutically acceptable salts and this may be preferred in some cases to increase solubility or dispersability, to reduce adverse side effects, etc.

The non-flavonoid antioxidant component in the topical composition is used in an amount of about 100 mg-50 gm, more preferably of about 250 mg-10 gm, and most preferably of about 500 mg-5 gm per one pound of topical base. Preferably, the non-flavonoid antioxidant component is superoxide dismutase, glutathione, or lipoic acid, more preferably, α-lipoic acid and, most preferably, DL-α-lipoic acid.

When the composition includes both vitamins A and D₃, they are preferably formulated together in a corn oil dispersion. Generally, each cubic centimeter (cc) or milliliter (mL) of the corn oil dispersion contains about 500,000 to about 2,000,000 IU of vitamin A and about 50,000 to about 200,000 IU of vitamin D₃. Preferably, every milliliter of the corn oil contains about 800,000 to about 1,200,000 IU of vitamin A and about 80,000 IU to about 120,000 IU of vitamin D₃. More preferably, the composition used in the invention contains about 1,000,000 IU and about 100,000 IU of vitamins A and D₃, respectively.

When vitamin A is administered, the dosage is preferably about 170 to about 360 IU per kg bodyweight, per day. More preferably, the dosage is about 214.3 to about 357.1 IU per kg bodyweight, per day. Still more preferably, the dosage is about 220 to about 340 IU per kg bodyweight, per day.

The amount of vitamin E per pound of topical base is an amount equivalent to the amount of vitamin E contained in about 0.5-5 cc of vitamin E acetate, more preferably of about 0.75-4.5 cc of vitamin E acetate, and most preferably of about 1-4 cc of vitamin E acetate. Each gram of vitamin E contains about 1000 IU of vitamin E.

About 11 to about 29 mg/kg bodyweight/day of ascorbyl palmitate may be administered. More preferably, about 14.3 to about 28.6 mg/kg bodyweight/day is administered.

The non-flavonoid antioxidants used in the composition of the present invention may include at least one of the compositions having vitamin C activity disclosed in U.S. Pat. Nos. 4,822,816 and 5,070,085 to Markham, which are also commonly called Ester-C®. The Ester-C® disclosed in U.S. Pat. Nos. 4,822,816 and 5,070,085 to Markham generally includes (a) an effective amount of a compound having Vitamin C activity. As used herein, the term, “compound having Vitamin C activity” means Vitamin C (L-ascorbic acid) and any derivatives thereof which exhibit antiscorbutic activity. Such derivatives include, for example, oxidation products, such as dehydroascorbic acid and edible salts of ascorbic acid such as, illustratively, calcium, sodium, magnesium, potassium and zinc ascorbates, esters of Vitamin C with organic and inorganic acids, such as L-ascorbic acid 2-0-sulfate, L-ascorbic acid 2-0-phosphate, L-ascorbic acid 3-0-phosphate, L-ascorbic acid 6-hexadecanoate, L-ascorbic acid monostearate, L-ascorbic acid dipalmitate and the like.

Metabolites of ascorbic acid and its derivatives include the aldonic acids, aldono-lactones, aldono-lactides and edible salts of aldonic acids. Preferably, the compound having Vitamin C activity includes one or more of these metabolites selected from L-threonic acid, L-xylonic acid and L-lyxonic acid. The presence of one or more of these metabolites in the compositions of the invention may provide an improvement in absorption and/or retention of Vitamin C or other therapeutically active compounds.

Studies (see Elmets, C. A. et al, J. Am. Acad. Dermatol., 44 (3); 425-32, March, 2001) have shown that green tea polyphenol or extract is effective in inhibiting erythema and preventing Langerhans cells from some forms of ultraviolet radiation damage.

The non-flavonoid antioxidants used in the composition of the present invention are selected not only for their antioxidant activity, but also based on other beneficial effects that particular compounds may provide. For example, a racemic mixture of α-lipoic acid not only has a strong antioxidant activity but also has a recycling effect on vitamins C and E, and thus is a preferred antioxidant for the present invention. In addition, α-lipoic acid can function in both lipid and non-lipid environments. The amount of α-lipoic acid used in the topical composition is in an amount of about 250 mg-2 gm, more preferably of about 400 mg-1.5 gm, and most preferably of about 500 mg-1 gm per one pound of topical base.

Similarly, vitamin E and its esters may contribute to an anti-cancer effect and may have beneficial effects on the skin. Vitamin E is optionally be included as one of the antioxidants in the topical composition.

Vitamin C and its esters are not only antioxidants, but also exhibit a strong combinatorial effect with vitamin E and its esters when used together. In fact, vitamin E and its esters, and vitamin C and its esters can mutually reinforce one another by a mechanism in which one antioxidant (reducing agent) acts as a regenerator for the oxidized form of the other. In addition, some of the antioxidants useful in the present invention are more active in a lipid environment whereas others are more active in a non-lipid environment. Accordingly, the composition of the present invention may include a combination of at least two antioxidants, with one being selected for its higher activity in a lipid environment and a second one being selected for its higher activity in a non-lipid environment.

Vitamin A (retinol or retinyl ester) may also have anti-cancer effects. In addition, vitamin A may also enhance the physiological mechanism of cell differentiation, inhibit malignant transformation, suppress tumor promotion and directly act against neoplastic cells. Vitamin A is also a fat-soluble material and thus is preferred for use due to this additional beneficial property. Preferably, vitamin A may be used in its ester forms, such as vitamin A palmitate, because the ester forms of vitamin A may be less irritating to the stomach.

Carotenoids such as β-carotene may also be included in the composition of the present invention as an antioxidant. Several carotenoids have shown beneficial effects for the present application, such as enhancement of immune response, inhibition of mutagenesis and/or reduction of induced nuclear damage. Carotenoids can also protect against photo-induced tissue damage. Some carotenoids, including β-carotene, quench highly reactive singlet oxygen under certain conditions and can block free radical-mediated reactions.

Another antioxidant that may be added to the topical composition disclosed herein is chlorophyllin and/or its salts, because chlorophyllin and its salts may exhibit beneficial effects such as an anti-cancer effect, and protection of DNA against ionizing radiation and other chemical mutagens, in addition to being a potent antioxidant. Chlorophyllin and its salts may be included in the composition of the present invention as part of the antioxidant. More preferably, chlorophyllin and its salts may be included in the composition of the present invention in amounts which, when administered to a patient according to a method of the present invention, provide a daily dosage between about 20 milligrams and about 500 milligrams. Chlorophyllin and its salts may be obtained from an alfalfa extract or extracted from silkworm feces. Chlorophyllin and its salts may also be purchased from common commercial sources such as Aldrich Chemical Company.

The antioxidant used in the composition of the present invention may include a combination of effective amounts of vitamin A or its esters, vitamin C or its esters, vitamin E and α-lipoic acid to achieve the beneficial effect of recycling vitamin C or its esters and vitamin E by α-lipoic acid.

Structurally similar derivatives of one or more of the aforementioned compounds, which exhibit antioxidant activity when applied to the patient, may also be employed. By “structurally similar derivatives” is meant derivatives that exhibit antioxidant activity and contain at least one significant, common structural element with the compound or material from which it is derived.

Antioxidant Enzymes

In another embodiment, the non-flavonoid antioxidant used in the composition of the present invention may include one or more antioxidant enzymes. The antioxidant enzymes useful in the present invention are those capable of scavenging radicals, promoting radical scavengers or preventing radical formation. The preferred antioxidant enzymes useful in the present invention include superoxide dismutase, catalase, glutathione peroxidase and methionine reductase. Other antioxidant enzymes with activities similar to those mentioned explicitly above, may also be used. In addition, one or more of the antioxidant enzymes may act in combination with one or more of the antioxidant compounds in the composition to, for example, scavenge free radicals and/or prevent cell damage in the skin. When the composition is formulated into a topical composition, the antioxidant enzyme used in the present invention is preferably skin-absorbable.

Compounds that Regulate Cell Differentiation and Cell Proliferation

Compounds that regulate cell differentiation and/or cell proliferation may also have antioxidant properties and be used as the antioxidant ingredient of the composition. The topical composition of the present invention may optionally include other compounds that regulate cell differentiation and/or cell proliferation. Such compounds may be selected from suitable compounds that have this activity. Suitable compounds that regulate cell differentiation and/or cell proliferation are those that do not induce significant, adverse side effects when administered to a patient in amounts that regulate cell differentiation and/or cell proliferation, and which do not react with one or more of the ingredients of the composition resulting in a substantial loss of activity of one or more of the ingredients. In one embodiment the compounds used for regulating cell differentiation and/or cell proliferation are those that occur naturally in the human body and/or materials that can be obtained from plants or animals which may be administered to humans in a safe and effective amount.

The compounds that regulate cell differentiation and/or cell proliferation used in the present invention inhibit or prevent cell differentiation or cell proliferation. In one embodiment the compounds that regulate cell differentiation and/or cell proliferation used in the present invention accomplish at least one of the following: maintenance of cellular homeostasis and normal cell metabolism, regulation of cell differentiation, induction of certain cancer cells to differentiate into normal cells, preferably by working in combination with vitamin A, maintenance of the epidermal permeability barrier, inhibition of cancer cell differentiation, and inhibition of cancer cell proliferation.

In one embodiment in order to formulate the compound that regulates cell differentiation and/or cell proliferation in the composition of the present invention it may be advantageous to use a dispersant. Suitable dispersants are known to persons skilled in the art. A particularly suitable dispersant for the compound that regulates cell differentiation and/or cell proliferation, is corn oil. Corn oil also has the advantage that it is a natural product. The amount of corn oil used is an amount sufficient to disperse the compound that regulates cell differentiation and/or cell proliferation.

Screening for Compounds that Regulate Cell Differentiation and/or Cell Proliferation

Methods for screening compounds that regulate cell differentiation and/or cell proliferation are well-known. For example, DiscoveRx Corporation at Fremont, Calif. markets a Hithunter™ tyrosine kinase assay to detect inhibitors of tyrosine kinase and tyrosine phosphatase which control or regulates cellular growth, proliferation and differentiation using β-galactosidase EFC activity. In this assay, inactive fragments of galactosidase, enzyme acceptor (EA) and enzyme donor (ED) complement to form active enzyme. Binding of an ED-conjugated peptide to an antibody inhibits complementation, while unlabeled peptide displaces the ED-conjugate. This results in increased β-galactosidase activity that is detected subsequently either chemiluminescence or long wavelength fluorescent substrates.

Hithunter™ tyrosine kinase assay has been developed to measure activity of the human insulin receptor, EGF receptor kinase domains and Src (EC 50=2.8 nM, 4.4 nM and 4.9 nM respectively). Hithunter™ tyrosine phosphatase activity was also measured using PTP 1B enzyme (EC 50=48 nM). Assay performance characteristics (Z′=0.5-0.7, CV=5-8%) and a simple two-step addition protocol make it ideal for HTS (high throughput screening).

Another exemplary method for screening compounds that regulate cell differentiation and/or cell proliferation is available from the Commercial Ventures & Intellectual Property Office at University of Massachusetts, Worcester, Mass. The method can be used to screen for cancer drugs and other drugs that inhibit or promote cell growth, cell death or cell differentiation for diseases involving ERb action, including prostate, breast and ovarian cancer, neurological disorders, osteoporosis and cardiovascular disease. In the method, the effect of any compound on ER-beta regulated cell growth/cell death/cell cycle arrest is determined by adding the compound to culture cells expressing the receptor and measuring alteration in expression levels of ER-beta regulated genes.

Cell Differentiation/Proliferation Compounds

Exemplary compounds that regulate cell differentiation and/or cell proliferation are vitamin D₃, vitamin D₃ analogs, compounds that may be converted or metabolized into vitamin D₃ in the human body, and metabolites thereof. Exemplary compounds that may be converted or metabolized into vitamin D₃ include common cholesterols illustrated below. The cholesterol illustrated below may be converted into Provitamin D when a hydrogen is removed from the number 7 carbon, which then forms a double bond with the number 8 carbon, in the second, or ‘B’ ring of the cholesterol molecule. The cholesterol is ‘oxidized’ (that is, an electron is removed with the hydrogen atom), so that the double bond is a consequence of 2 mutually shared electrons between carbons 7 and 8.

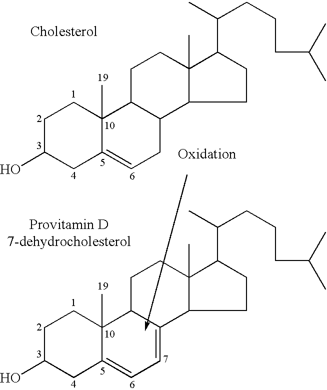

Provitamin D may be converted to Vitamin D₃ by the action of ultraviolet light through human shin. In this reaction, the B ring of the sterol molecule is opened.

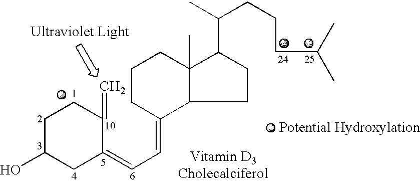

Cholecalciferol, which is Vitamin D₃, may be further converted into another vitamin D intermediate, 25-hydroxycholecalciferol, in the liver by mitochondrial hydroxylase, in the presence of NADPH, and molecular oxygen.

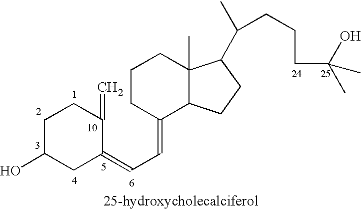

When more active vitamin D₃ is required, 25-hydroxycholecalciferol is transported to the kidney where a new hydrolase enzyme is synthesized. This enzyme introduces another hydroxyl group at position 1, and the bioactive form of Vitamin D₃, calcitriol, is produced.

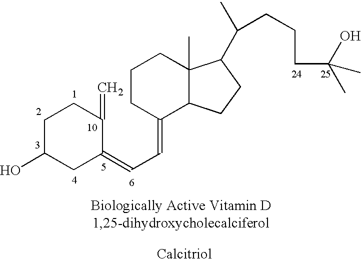

Exemplary vitamin D₃ analogs include 1(S), 3(R)-dihydroxy-20(R)-(1-ethoxy-5-ethyl-5-hydroxy-2-heptyn-1-yl)-9,10-seco-pregna-5(Z), 7(E), 10 (19)-triene. Exemplary vitamin D₃ metabolites include 1,25-dihydroxyvitamin D₃. Also, pharmaceutically acceptable salts of the compounds that regulate cell differentiation and/or cell proliferation may be employed. In one embodiment a compound that regulates cell differentiation and/or cell proliferation is vitamin D₃.

The compound(s) that regulates cell differentiation and/or cell proliferation is used in an amount that is safe and effective to regulate cell differentiation and/or cell proliferation when topically applied to a patient in the topical composition of the present invention. When a composition including vitamin D₃ or derivative or metabolite of vitamin D₃ is administered, the vitamin D₃ or derivative or metabolite of vitamin D₃ is used in a safe and effective amount. More preferably, an amount of about 6 to about 14.3 IU per kg of body weight of the patient for each administration. More preferably, an amount of about 8 to about 14.3 IU per kg body weight of the patient, and, still more preferably, an amount of about 10 to about 13 IU is employed per kg of body weight of the patient, is administered.

Other Optional Ingredients

The composition of the present invention may further include selenium and/or a compound containing selenium. Selenium is known to be able to prolong the lifespan of a person exposed a severe dose of harmful radiation, and to reduce the potential occurrence of leukemia and other malignancies in that person. Selenium may be included in the composition of the present invention in a safe and effective amount. Preferably, the daily dosage should be between 5 micrograms and 200 micrograms. Selenium may be included in the composition in such an amount that when the composition is applied to a human according to a method of the present invention, the daily dosage should be between 10 micrograms and 100 micrograms.

The topical compositions of the present invention may further include an organic germanium compound such as carboxy ethyl sesquioxide of germanium or spirogermanium. Organic germanium compounds are known to protect human cells from radiation damage. For example, controlled experiments have also shown that Ge-132 reduces mutations in E. coli due to γ-radiation by twenty-fold (see Mochizuki and Kada, Antimutagenic effect of Ge-132 on γ-ray-induced mutations in E. coli B/r WP2 trp-. 42(6) Int. J. Radiat. Biol, 653-59 (1982)). Germanium oxide has been shown to reduce the mutation rate in Salmonella typhimurium induced by Trp-P-2 (3-amino-1-methyl-5H-pyrido(4,3-b)indole), by 40-67 folds (see Kada, Mochizuki, and Miyao, Antimutagenic Effects of Germanium Oxide on Trp-P-2 Induced Frameshift Mutations in Salmonella Typhimurium TA98 and TA 1538, 125 Mutation Research, 145-51 (1984)). One or more organic germanium compounds may be included in the composition of the present invention in a safe and effective amount. Preferably, the daily dosage of the germanium compound will be between 25 milligrams and 500 milligrams. Preferably, the organic germanium may be included in the composition in such an amount that when the composition is administered to a human according to a method of the present invention, the daily dosage of the germanium compound will be between 50 milligrams and 200 milligrams, and, most preferably, about 100 milligrams.

Alternatively, Siberian ginseng may be added to the topical compositions of the present invention in the form of one or more of Siberian ginseng roots, Siberian ginseng powder, or extracts thereof which may contain one or more of the active ingredients of the Siberian ginseng. Siberian ginseng (Eleutherococcus senticosus) has been shown to have restorative effects on the functions of bone marrow damaged by exposure to radiation. The active ingredients of Siberian ginseng generally include eleutherosides A, B, B1, C, D and E; triterpenoid saponins; eleutherans A, B, C, D, E, F and G; and equivalents thereof. Siberian ginseng extract may be included in the composition of the present invention in such an amount that when the composition is applied to a human according to a method of the present invention, the daily dosage of the Siberian ginseng extract will be between 25 milligrams and 500 milligrams. Siberian ginseng extract may be included in the composition in a safe and effective amount. Preferably, the daily dosage of Siberian ginseng extract should be between 50 milligrams and 150 milligrams, and, most preferably, the daily dosage of the Siberian ginseng extract will be about 100 milligrams. If Siberian ginseng is used in a different form in the composition of the present invention, a skilled person should be able to adjust the amount being used accordingly based on the dosages for the Siberian ginseng extract given above.

Alternatively, the compositions of the present invention may include Korean ginseng (panax ginseng) and/or American ginseng (panax quinquefolius), in the form of roots, powder or an extract. Korean and/or American ginseng may prompt recovery of hemateikon and splenal weight and cause improvement of thrombocyte cells. This product is commercially available as Korea Insam. The daily dosage for Korean and/or American ginseng is the same as for Siberian ginseng. A skilled person is able to adjust the dosage of the Korean and/or American ginseng for different physical forms, i.e. root, powder or extract. Of course, mixtures of one or more of Siberian ginseng, Korean ginseng and American ginseng and/or extracts of one or more of these ginseng types may also be employed.

In one embodiment of the present invention, the compositions are substantially free of cinnamic acid derivatives of the formula:

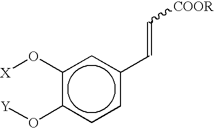

wherein the groups X, Y and R, independently of one another, can be chosen from the group consisting of H and branched or unbranched alkyl having 1-18 carbon atoms, acids thereof, and physiologically tolerated salts thereof.

Benefits of the Topical Composition

Compositions in accordance with the present invention may provide one or more of the following localized or systemic beneficial effects to a mammal when topically applied in an effective amount: antioxidant properties, free radical scavenging, transition metal chelation, nitric oxide stabilization, anti-inflammatory activity, relief of pain, burning, tingling, electrical sensations and/or hyperalgesia, increased microcirculation, nitric oxide stabilization, promotion of healing of skin ulcers and lesions, protein kinase C inhibition, decreased oxidative stress, an anti-inflammatory effect, protection against radiation damage, blockage of the formation of leukotrienes, stabilization of cell membranes, and regulation of cell differentiation and/or cell proliferation, protection of mitochondrial membranes, reduction of cell damage, especially damage to DNA molecules, and may play a role in the repair and regeneration of damaged cells.

Compositions in accordance with the invention can provide additional effects of improving the appearance of the skin. Skin appearance may be adversely affected by ionizing radiation, for example in radiation dermatitis, or by other causes unrelated to the exposure to ionizing radiation. One or more of the following beneficial properties may be realized when compositions of the invention are topically applied in an effective amount: reducing or preventing redness of skin, reducing or preventing discoloration of skin, beautifying skin and/or hair, improving appearance of skin and/or hair, promoting attractiveness of skin and/or hair, cleansing skin and/or hair, removing dead or damaged skin or skin cells from skin and/or hair and moisturizing skin and/or hair.

Treating, Reducing or Preventing an Adverse Effect of Radiation

In another aspect, the present invention relates to a method of preventing, reducing or treating at least one adverse effect of radiation by the topical application of an amount of a composition, which includes a mixture of at least two ingredients including at least one flavonoid and at least one non-flavonoid anti-oxidant.

In the preferred embodiment, the method of the present invention involves the topical application of a composition of the present invention to a mammal that may be potentially exposed to ionizing radiation, is in the process of being exposed to ionizing radiation, or has already been exposed to ionizing radiation. Preferably, the ionizing radiation is selected from alpha-radiation, beta-radiation, gamma ray radiation, and x-ray radiation. The effective amount of the topical composition to be applied will vary depending on such factors as the characteristics of the patient, the particular mode of administration, the activity of the particular non-carrier ingredients employed, the age, bodyweight, general health, sex and diet of the patient, the time of application, the particular combination of ingredients employed, the total content of the non-carrier ingredients of the topical composition, and the severity of the adverse effect of radiation, radiation injury or expected radiation exposure. It is within the skill of the person of ordinary skill in the art to account for these factors to provide a suitable dosage and treatment regimen for a standard 70 kg adult, described below.

The above-mentioned composition is applied to the afflicted area of the skin in an amount of approximately at least 0.5 mL per 100 cm² per day, preferably at least about 1 mL per 100 cm² per day, and more preferably about 15 mL per 100 cm² per day. Preferably, the daily dosage does not exceed about 35 mL per 100 cm² per day.

The present invention also relates to a method for treating adverse effects on the appearance of the skin and/or hair caused by exposure to ionizing radiation such as, for example, redness, discoloration, dryness. In this aspect, the invention can reduce, treat or prevent adverse effects of ionizing radiation by, for example, reducing or preventing redness of skin, reducing or preventing discoloration of skin, beautifying skin and/or hair, improving appearance of skin and/or hair, promoting attractiveness of skin and/or hair, cleansing skin and/or hair, removing dead or damaged skin or skin cells from skin and/or hair and moisturizing skin and/or hair.

Application of the Topical Composition

For prevention, reduction or treatment of at least one adverse effect of radiation, the topical composition is preferably applied to the skin before potential exposure to radiation. In another embodiment of the invention the topical composition is applied to the skin at least once twenty-four hours before the start of the potential radiation exposure, and three times (e.g., morning, noon and bedtime) in the 24-hour period immediately before the potential radiation exposure. For each application, an amount of the composition is applied which is sufficient to cover the area of the skin to be potentially exposed to radiation with a thin layer of the topical composition. The topical composition should be rubbed into the skin until little or no residue remains on the skin.

In a method for treating or reducing radiation injury, an effective amount of the topical composition of the invention is applied one to six times daily, as needed, to an area of skin inflicted with radiation injury during and/or after radiation exposure. In a preferred method, a thin layer of the topical composition is preferably applied to the inflicted area of skin, as needed, and rubbed into the skin until little or no residue remains on the skin.

Acceptable Carriers

The acceptable topical carrier used in the present invention may be a carrier suitable for use as a carrier for topical compositions. The non-carrier ingredients, which may include flavonoids, non-flavonoid antioxidants, compounds that regulate cell differentiation and/or cell proliferation, selenium and /or a selenium compound, organic germanium compounds, components of ginseng, as well as inositol, other B-complex vitamins, and anti-inflammatories such as γ-linolenic acid, are dissolved, dispersed and/or suspended in the topical composition. Exemplary topical carriers may include bases for creams, ointments, lotions, pastes, jellies, sprays, aerosols, bath oils, and other topical carriers, which accomplish direct contact between the active ingredients of the topical composition of the present invention and the pores of the skin. Preferably, the acceptable topical carrier may make up more than about 80%, and more preferably about 80-95% w/w of the total composition. In some cases, it may be necessary to dissolve one or more the active ingredients in an appropriate solvent such as ethanol or DMSO (dimethylsulfoxide), and the like, to facilitate the incorporation of the one or more active ingredients into the topical composition or the acceptable topical carrier.

In one embodiment the topical carrier may contain, as the base, at least a hydrophilic ointment base, panthenol or a panthenol derivative, and a dispersant, if needed, to disperse one or more insoluble or partially insoluble active ingredients in the carrier (infra). Another embodiment of the topical carrier of the present invention employs hydroxymethyl cellulose as the base and may contain ingredients contained in the carrier described below other than the hydrophilic ointment base.

Yet another acceptable topical carrier may include, as the base, a solution of an acrylic copolymer in a non-aqueous solvent system, which mainly contains polyethylene glycol such as methoxy polyethylene glycol 550 (MPEG). A particular preferred MPEG is SENTRY CARBOWAX MPEG 550 sold by Union Carbide, which is a food/pharmaceutical/cosmetic grade material. Polyethylene glycols are generally non-toxic, water-soluble polymers that are fully biodegradable. In the solution, the acrylic copolymer would preferably be present in a concentration range of 3-6% by weight. In one embodiment the acrylic copolymer has a molecular weight of more than 20,000. In another embodiment the acrylic copolymer has a molecular weight of more than 100,000 so that it will not be systematically absorbed by the human body or skin. Components of the carrier material other than the hydrophilic ointment base may also be employed in this carrier material.

Suitable hydrophilic ointment bases are known to persons skilled in the art. Exemplary hydrophilic ointment bases suitable for use in the present invention are non-U.S.P. hydrophilic ointment bases such as those made by Fougera, Inc. Sufficient hydrophilic ointment base is employed to act as a topical carrier for the active or non-carrier ingredients of the topical composition. Typically, the hydrophilic ointment base will make up more than about 80% of the total composition, and more preferably about 80-95% of the composition is the hydrophilic ointment base. The hydrophilic ointment base functions as a topical carrier and enhances penetration into the skin. Similar proportions of the hydroxymethyl cellulose-based carrier or acrylic copolymer solution based carrier may also be employed.

The panthenol or panthenol derivatives useful in the present invention include at least D-panthenol, DL-panthenol and mixtures thereof. This component of the topical carrier has skin moisturizing properties and acts as a quick, deep penetrating component of the topical carrier that helps deliver the non-carrier ingredients through the skin to the area to be treated and may also impart a healing effect to damaged tissue. The amount of panthenol or panthenol derivative to be employed is from about 0.25 to about 10 weight percent, more preferably from about 0.5 to about 5 weight percent and most preferably from about 1 to about 2 weight percent, based on the total weight of the topical composition.

The topical carrier of the present invention may also include additional ingredients such as other carriers, moisturizers, humectants, emollients, dispersants, radiation blocking compounds, particularly UV-blockers, as well as other suitable materials that do not have a significant adverse effect on the activity of the topical composition. Additional ingredients for inclusion in the topical carrier are sodium acid phosphate moisturizer, witch hazel extract, glycerine humectant, apricot kernal oil emollient, AJIDEW NL-50 NaPCA (50% aqueous solution) and corn oil dispersant.

The topical composition of the present invention may also be employed to facilitate wound healing, for the treatment of skin cancer and/or one or more symptoms thereof, or as a topical composition for protecting skin from the harmful or adverse effects of ionizing radiation, such as radiation breakdown or radiation recall dermatitis.

Preparation of the Topical Composition

The topical composition of the present invention is preferably made by cold compounding. This may be an important feature of the invention if one or more of the compounds employed in the topical composition are sensitive to heat or other types of energy in which case the activity of the topical composition may be detrimentally affected as a result of the formulation of the topical compositions in another manner. Thus, the ingredients of the topical composition the present invention are preferably mixed together, without heating using a sufficient amount of the topical carrier to provide a substantially homogeneous cream or ointment. It may be necessary to dissolve, disperse or suspend one or more of the ingredients prior to cold compounding in order to ensure substantially homogeneous distribution of the non-carrier or active ingredients in the topical composition.

A acceptable topical carrier of the invention can be made using the following ingredients, all based on use of one pound of the topical base. 25-35 parts of a 50% aqueous solution of sodium acid phosphate moisturizing agent, 5-10 parts of D- or DL-panthenol, 5-10 parts of glycerine, 1-3 parts of apricot kernal oil and 10-20 parts of witch hazel extract. The witch hazel extract is used in an amount of about 2.5-40 cc, more preferably of about 5-30 cc, and most preferably of about 10-20 cc per pound of topical base. The glycerine humectant is used in an amount of about 2-20 cc, more preferably of about 3.5-15 cc, and most preferably of about 5-10 cc per pound of topical base. The apricot kernel oil is used in an amount of about 0.5-5 cc, more preferably of about 0.5-4 cc, and most preferably of about 1-3 cc per pound of topical base. The AJIDEW NL-50 NaPCA (50% aqueus solution) is used in an amount of about 15-45 cc, more preferably of about 20-40 cc, and most preferably of about 25-35 cc per pound of topical base.

One embodiment of the topical composition of the present invention comprises a particularly preferred combination of ingredients, which includes green tea or green tea extract, a non-flavonoid antioxidant, and at least one flavonoid. Optionally, one or more of the optional ingredients of the topical composition such as glycerin, witch hazel extract, vitamins A and E and/or the ascorbyl palmitate can be reduced or eliminated from a particular topical composition, if desirable, or larger amounts of one type of component, i.e. an antioxidant, can be employed while reducing the amount of another component of the same type or having a similar activity.

The invention will now be further illustrated by the following example. This is one example of the invention disclosed herein and is not meant to limit the invention.

### EXAMPLE 1

A topical composition including a mixture of an hydrophilic ointment base, sodium acid phosphate moisturizing agent, a witch hazel extract carrier, glycerine, apricot kernal oil and DL-panthenol, as the acceptable carrier and vitamins A and D₃, ascorbyl palmitate, α-lipoic acid, quercetin and vitamin E acetate was prepared by cold compounding. The formulation of the topical composition is given in Table 1.

The topical composition was prepared by first placing the hydrophilic ointment base in a stainless steel bowl and mixing briskly until the ointment becomes creamy. Then, the sodium acid phosphate, panthenol, ascorbyl palmitate, glycerine, apricot kernal oil, vitamins A and D₃, quercetin, witch hazel extract, vitamin E acetate and α-lipoic acid were added in that order. After each ingredient was added, mixing was continued until all traces of dry ingredients disappeared and a substantially homogeneous mixture was obtained. The final color should be a consistent yellow and the cream should have the consistency of cake frosting. The mixture was then placed in a sterile container. All containers which contact the topical composition during mixing must also be sterilized with, for example, zephiran chloride or a Clorox solution such as betadine.

This composition was topically applied, under the supervision of a physician, to several patients a day before undergoing radiation therapy treatment. The administration of the topical composition was carried out by applying a thin film of the composition to the areas of the skin to be exposed to radiation. The topical composition was applied three times during that day in the morning, noon and at bedtime. All of the patients administered with the topical composition of the present invention experienced much less severe radiation dermatitis after radiation therapy than patients who were not treated with the topical composition of the invention. The effects noted by the patients included reductions in burning, irritation and redness in the areas of skin that were treated.

TABLE 1

| | |
|---|---|
| 1 lb. | Hydrophilic Ointment Base (Non-USP) |
| 25 cc | AJIDEW NL-50 NaPCA (50% Aqueus Solution) |
| 5 cc | DL-Panthenol |
| 5 cc | Glycerin (USP) |
| 3 cc | Apricot Kernel Oil |
| 6 cc | Vitamin A in corn oil (1 million 1 u/gm) and Vitamin |
| | D₃ in corn oil (100,000 1 u/gm) |
| 12 cc | Witch Hazel Extract; 14% Alcohol |
| 2 cc | Vitamin E Acetate (1 gram = 1000 IU) |
| 2 gm | Ascorbyl Palmitate |
| 2 gm | Quercetin Dihydrate |
| 1 gm | DL-α-lipoic acid |
| 500 mg | Green Tea |
| 500 mg | Rutin |
| Yield: 16-18 ozs. | |

### EXAMPLE 2

A composition in accordance with Example 1, except that the 500 mg of green tea were not included, was administered under supervision by a doctor for the purpose of improving the cosmetic appearance of skin exposed to radiation. The composition was topically administered to two patients prior to, during and after undergoing therapeutic radiation treatment. The composition was administered by topical application to the skin 1-2 times daily, as needed, starting 2-3 days prior to the radiation exposure for the purpose of improving the cosmetic appearance of the skin. Each application was up to about 5 cc of the ointment to the area of skin to be exposed.

The first patient was exposed to 30 grays of Bolus radiation with proton beam in a single day. The composition was applied again approximately 3 hours prior to exposure and 1-2 times daily after exposure from 2-3 additional days. This patient developed only a mild reddening of the skin, which was highly unexpected due to the very high dose of radiation to which this patient was exposed.

The second patient was exposed to a radiation dosage of about 1.5 gray per day, each day for a period of 60 days. The ointment was applied again approximately 3 hours prior to the first exposure and 1-2 times daily throughout the 60-day treatment period. The ointment was also applied 1-2 times daily for 2-3 days after the last radiation exposure. Again, the patient developed only a mild reddening of the skin, which was highly unexpected due to the very high dose of radiation to which this patient was exposed over the 60-day period.

The foregoing detailed description of the invention and examples are not intended to limit the scope of the invention in any way and should not be construed as limiting the scope of the invention. The scope of the invention is to be determined from the claims appended hereto.

What is claimed is:

1. A method for the reduction, treatment or prevention of at least one adverse effect of ionizing radiation in a mammal comprising the step of applying to an area of skin of the mammal which has been, is being or will be exposed to radiation, an amount of a topical composition which comprises a mixture of at least one non-flavonoid antioxidant and at least one flavonoid, formulated in an acceptable carrier for a topical composition, which is effective, when topically applied, to reduce, treat or prevent an adverse effect of ionizing radiation.

2. The method of claim 1, wherein the adverse effect of ionizing radiation is deterioration in the appearance of the skin of the mammal.

3. The method of claim 1, wherein the topical application of said topical composition provides an effect selected from the group consisting of reducing or preventing discoloration of skin, beautifying skin and/or hair, improving appearance of skin or hair, promoting attractiveness of skin and/or hair, cleansing skin or hair, removing dead or damaged skin or hair and moisturizing skin or hair.

4. The method of claim 1, wherein said step of applying comprises rubbing, pouring, spraying or sprinkling.

5. The method of claim 1, wherein the adverse effect of ionizing radiation comprises radiation injury.

6. The method of claim 5, wherein the radiation injury is selected from the group consisting of free radical damage, inflammation, pain, burning, tingling, hyperlagesia, a decrease in microcirculation, ulcers, lesions, destabilization of cell membranes, cell damage, and DNA damage.

7. The method of claim 1, wherein the adverse effect of ionizing radiation is localized.

8. The method of claim 1, wherein the adverse effect of ionizing radiation is systemic.

9. The method of claim 1, wherein the ionizing radiation originates from a therapeutic or medical source.

10. The method of claim 1, wherein the ionizing radiation originates from accidental or malicious radiation exposure.

11. A method as claimed in claim 1, wherein said radiation is selected from the group consisting of α-radiation, β-radiation, γ radiation and x-ray radiation.

12. The method of claim 1, wherein the topical composition further comprises at least one component of green tea or an acceptable salt thereof.

13. The method of claim 12, wherein the at least one component of green tea is selected from epigallocatechin-3-gallate, (-)-epigallocatechin and (-)-epicatechin or an acceptable salt thereof.

14. The method of claim 1, wherein said at least one non-flavonoid antioxidant is selected from the group consisting of: lipoic acid, ascorbyl palmitate, ascorbic acid, vitamin A, vitamin A ester, vitamin E, vitamin E acetate, carotenoids, green tea polyphenols, glutathione, structurally similar derivatives thereof which exhibit antioxidant activity, and acceptable salts thereof.

15. The method of claim 1, wherein said at least one non-flavonoid antioxidant is selected from the group consisting of: vitamin A, vitamin A esters, vitamin E, vitamin E esters, lipoic acid, carotenoids, β-carotene, chlorophyllin, coenzyme Q10, glutathione, L-dopa, cysteine, N-acetyl cysteine, cystine, pangamic acid (dimethyl glycine), taurine, tyrosine, beta-1,3-glucan, germanium, alpha-hydroxy acids, phytic acid (inositol hexaphosphate), caffeic acid (3,4-dihydroxy-cinnamic acid), ellagic acid, 3,3′, 4-tri-o-methyl ellagic acid, ferulic acid, gallic acid, gamma-oryzanol, resveratrol (trans-3,5,4′-trihydroxystilbene), zingerone (4-(4-hydroxy-3-methoxyphenyl)-2-butanone), nordihydroguaiaretic acid, pyrroloquinoline quinone, allicin, dithiolthiones, glucosilinates, S-allyl-L-cysteine, tocotrienols, camosol, indole, polyphenols, and salts and solvates thereof.

16. The method of claim 1, wherein said at least one non-flavonoid antioxidant comprises at least one antioxidant enzyme.

17. The method of claim 16, wherein said antioxidant enzyme is selected from superoxide dismutase, catalase, glutathione peroxidase and methionine reductase.

18. The method of claim 16, wherein said antioxidant enzyme can exhibit an effect selected from scavenging radicals, promoting radical scavengers or preventing radical formation.

19. The method of claim 16, wherein said antioxidant enzyme is skin-absorbable.

20. The method of claim 1, wherein the at least one flavonoid is selected from the group consisting of: 1,2,3,6-tetra-o-gallyol-β-d-glucose; 2′o-acetylacetoside; 3,3′, 4-tri-o-methyl-ellagic acid; 6,3′, 4′-trihydroxy-5,7,8-trimethoxyflavone; 6-hydroxy-luteolin; 6-hydroxykaempferol-3,6-dimethyl ether; 7-o-acetyl-8-epi-loganic acid; acacetin; acetoside; acetyl trisulfate quercetin; amentoflavone; apigenin; apiin; astragalin; avicularin; axillarin; baicalein; brazilin; brevifolin carboxylic acid; caryophyllene; chrysin-5,7-dihydroxyflavone; chrysoeriol; chrysosplenol; chrysosplenoside-a; chrysosplenoside-d; cosmosiin; δ-cadinene; dimethylmussaenoside; diacerylcirsimaritin; diosmetin; dosmetin; ellagic acid; ebinin; ethyl brevifolin carboxylate; flavocannibiside; flavosativaside; genistein; gossypetin -8-glucoside; haematoxylin; hesperidine; hispiduloside; hyperin; indole; iridine; isoliquiritigenin; isoliquiritin; isoquercitrin; jionoside; juglanin; kaempferol-3-rhamnoside; kaempferol-3-neohesperidoside; kolaviron; licuraside; linariin; linarin; lonicerin; luteolin; luetolin-7-glucoside; luteolin-7-glucoside; luetolin-7-glucoronide; macrocarpal-a; macrocarpal-b; macrocarpal-d; macrocarpal-g; maniflavone; methy scutellarein; naringenin; naringin; nelumboside; nepetin; nepetrin; nerolidol; oxyayanin-a; pectolinarigenin; pectolinarin; quercetagetin; quercetin; quercimertrin; quercitrin; quercitryl-2″ acetate; reynoutrin; rhamnetin; rhoifolin; rutin; scutellarein; sideritoflavone; sophoricoside; sorbarin; spiraeoside; trifolin; vitexin; wogonin; and acceptable salts thereof.

21. The method of claim 1, wherein the at least one flavonoid is selected from the group consisting of quercetin, rutin, myricetin, kaempferol, myrecetrin, curcuminoids and acceptable salts thereof.

22. The method of claim 1, wherein the at least one flavonoid comprises quercetin or an acceptable salt thereof.

23. The method of claim 1, wherein the at least one flavonoid comprises rutin or an acceptable salt thereof.

24. The method of claim 1, wherein the at least one non-flavonoid antioxidant comprises lipoic acid or an acceptable salt thereof.

25. The method of claim 24, wherein the lipoic acid is DL-α-lipoic acid.

26. The method of claim 22,wherein the at least one flavonoid further comprises rutin.

27. The method of claim 1, wherein the at least one non-flavonoid antioxidant comprises vitamin E.

28. The method of claim 1, wherein the at least one non-flavonoid antioxidant comprises vitamin A.

29. The method of claim 1, wherein the at least one non-flavonoid antioxidant comprises ascorbyl palmitate.

30. The method of claim 2, wherein said topical composition further comprises at least one compound that inhibits at least one of cell differentiation and cell proliferation.

31. The method of claim 30, wherein the at least one compound that inhibits at least one of cell differentiation and cell proliferation comprises a compound selected from the group consisting of vitamin D₃, 1(S), 3(R)-dihydroxy-20(R)-(1-ethoxy-5-ethyl-5-hydroxy-2-heptyn-1-yl)-9, 10-seco-pregna-5(Z), 7(E), 10 (19)-triene, 1, 25-dihydroxyvitamin D₃, and pharmaceutically acceptable salts thereof.

32. The method of claim 2, wherein the composition further comprises one or more ingredients selected from the group consisting of selenium and selenium compounds.

33. The method of claim 2, wherein the composition further comprises one or more ingredients selected from the group consisting of organic germanium, Korean ginseng, an extract of Korean ginseng, American ginseng, an extract of American ginseng, Siberian ginseng and an extract of Siberian ginseng.

34. The method of claim 1, wherein the non-flavonoid antoioxidant compound is vitamin C or a compound having vitamin C activity.

35. The method of claim 34, wherein the compound having vitamin C activity is selected from dehydroascorbic acid, calcium ascorbate, sodium ascorbate, magnesium ascorbate, potassium ascorbate zinc ascorbate, and Vitamin C esters.

36. The method of claim 35, wherein the vitamin C ester is selected from L-ascorbic acid 2-o-sulfate, L-ascorbic acid 2-o-phosphate, L-ascorbic acid 3-o-phosphate, L-ascorbic acid 6-hexadecanoate, L-ascorbic acid monostearate, and L-ascorbic acid dipalmitate.

37. The method of claim 1, said topical composition comprising quercetin, rutin, DL-γ-lipoic acid, vitamin E, vitamin A and ascorbyl palmitate.

38. A method for improving the appearance of skin and or hair comprising the step of applying to the skin of a mammal before during or after exposure to ionizing radiation an appearance improving amount of a topical composition which comprises at least one non-flavonoid antioxidant and at least one flavonoid, formulated in a carrier acceptable for a topical composition.

39. The method of claim 38, wherein improving the appearance of skin and or hair is selected from the group consisting of reducing or preventing redness of skin, reducing or preventing discoloration of skin, beautifying skin and/or hair, improving appearance of skin and/or hair, promoting attractiveness of skin and/or hair, cleansing skin and/or hair, removing dead or damaged skin or skin cells from skin and/or hair and moisturizing skin and/or hair.

40. The method of claim 38, wherein the step of applying comprises rubbing, pouring, spraying or sprinkling.
